(19) United States
(12) Patent Application Publication
(10) Pub. No.: US 2012/0256092 A1
(43) Pub. Date: Oct. 11, 2012

(54) CT SYSTEM FOR USE IN MULTI-MODALITY IMAGING SYSTEM

(75) Inventor: Yulim Zingerman, Netanya (IL)

(73) Assignee: General Electric Company, Schenectady, NY (US)

(21) Appl. No.: 13/081,366

(22) Filed: Apr. 6, 2011

(51) Int. Cl. A61B 6/03; G01T 1/166

(52) U.S. Cl. 250/363.03; 378/4; 250/363.04

(57) ABSTRACT

A computed tomography (CT) imaging system is disclosed. The CT imaging system may be used in a multi-modality imaging context or other context. In one embodiment, the CT imaging system provides for both fast rotation of the rotating X-ray source and detection components and low dose of X-rays generated by the source providing several clinical and economic benefits such as low dose and sufficient image quality and no or insignificant investment in room shielding associated with diagnostic CT dose.

### BACKGROUND OF THE INVENTION

The subject matter disclosed herein relates to a multi-modality imaging system employing computed tomography (CT) and, more particularly to single photon emission computed tomography (SPECT) or positron emission tomography (PET) systems combined as part of a SPECT/CT or PET/CT system.

Non-invasive imaging broadly encompasses techniques for generating images of the internal structures or regions of a person that are otherwise inaccessible for visual inspection. One of the best known uses of non-invasive imaging is in the medical arts where these techniques are used to generate images of organs and/or bones inside a patient which would otherwise not be visible. One class of medical non-invasive imaging modalities is based on the generation of structural images of internal structures which depict the physical arrangement of the imaged region. One example of such a modality is computed tomography (CT), which is based on the differential transmission of X-rays through the patient as seen from numerous radial views about the patient. In CT, the acquired X-ray transmission data may be used to generate three-dimensional volumes of the imaged region.

While structural imaging modalities generate images of the physical or anatomical arrangement of an internal region of interest of the patient, functional imaging modalities generate images reflecting the chemical composition or metabolic activity of the internal region of interest. One example of such a functional imaging modality is single-photon emission computed tomography (SPECT). In SPECT imaging, gamma rays are generated by a radioactive tracer introduced into the patient. Based on the type of metaboland, sugar, or other compound into which the radioactive tracer is incorporated, the radioactive tracer is accumulated in different parts of the patient and measurement of the resulting gamma rays can be used to localize and image the accumulation of the tracer. For example, tumors may disproportionately utilize glucose or other substrates relative to other tissues such that the tumors may be detected and localized using radioactively tagged deoxyglucose.

The different properties of structural and functional imaging may be combined to provide more information to a diagnostician than either modality alone. For example, in the case of combined SPECT/CT scanners, a clinician is able to acquire both SPECT and CT image data that can be used in conjunction to detect tumors or to evaluate the progression of a tumor. However, due to differences in the manner in which SPECT and CT systems operate, e.g., the physical phenomena measured and the manner in which measurement is accomplished, it may be difficult to design a combined modality imaging system that provides the desired functionality and performance with respect to each different imaging modality.

Further, certain of the structural modalities, such as CT, may utilize X-rays or other forms of radiation. In certain countries, regulations or best practices may limit the X-ray dose that may be experiences outside the room containing the imaging system, such as to not exceed 0.02 millisievert/week. To meet these requirements, the walls of a room housing such a system may be shielded (such as with lead plating having a thickness of 2 mm or more), which can add substantially to the cost of constructing a facility for housing such a system. Further, use of such shielding can environmental and recycling issues due to the presence of lead.

### BRIEF DESCRIPTION OF THE INVENTION

The present invention provides for a combined SPECT/CT imaging system that addresses problems that may be found in existing systems. In one embodiment, the present SPECT/CT system utilizes a distinct CT subsystem in which the CT detector components rotate independent of the gamma detectors of the SPECT subsystem and, in one implementation rotate at rotation speed greater than 30 RPM, and preferably about 60 rotations per minute (RPM) or above, typically the same or faster than the corresponding gamma detection components. Further, in one such implementation, the CT subsystem operates at a low dose (i.e., at a limited mAs and/or with a suitable bowtie filter). In embodiments where the CT subsystem operates at a low dose, the SPECT/CT system and/or the surrounding environment or room may use no or reduced shielding or radiation protection, in contrast to the higher level of shielding and/or protection that is typically associated with higher dose (e.g., diagnostic) CT systems. Further, the CT subsystem of the present SPECT/CT system may have a reduced footprint with respect to other conventional SPECT/CT systems.

In accordance with one aspect of the present disclosure, a dual-modality imaging system is provided. The dual-modality imaging system includes a nuclear medicine imaging subsystem comprising a gamma ray detection component suitable for acquiring functional image data. The dual-modality imaging system also includes a computed tomography (CT) subsystem suitable for acquiring structural image data. The CT subsystem comprises a gantry housing an X-ray source and an X-ray detector that are configured to rotate with respect to the gantry. The X-ray source and the X-ray detector rotate above 30 revolution per minute (RPM) during operation. The X-ray source operates at a current level below 30 mA during operation, such as between 10 mA and 30 mA.

In accordance with another aspect, a dual-modality imaging method is provided. In accordance with the method a set of functional image data is acquired using a nuclear medicine imaging subsystem of a dual-modality imaging system. A set of computed tomography (CT) imaging data is acquired using a CT imaging subsystem. A detector of the CT subsystem rotates at least above 30 revolutions per minute (RPM), such as about or above 60 RPM and an X-ray source of the CT subsystem operates at a current between about 10 mA and about 30 mA during acquisition of the set of CT imaging data. A localization image or attenuation map is generated using the set of CT imaging data.

In accordance with a further aspect, a CT imaging system is provided. The CT imaging system includes a gantry and an X-ray detector and X-ray source configured to rotate about the gantry. The X-ray source operates at a current level of between about 10 mA and about 30 mA during operation. The X-ray source and the X-ray detector during operation rotate about the gantry at least above 30 revolutions per minute RPM, such as at or above 60 RPM.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a side view of a SPECT/CT imaging system in accordance with aspects of the present disclosure in a room having shielding;

FIG. 2 depicts a side view of a SPECT/CT imaging system in accordance with aspects of the present disclosure in a room having no or reduced shielding;

FIG. 3 depicts a front-view of a CT subsystem for use in conjunction with the SPECT/CT imaging system of FIGS. 1 and 2; and

FIG. 4 is a cross-sectional view the CT subsystem of FIG. 3 taken along sight line 4.

### DETAILED DESCRIPTION OF THE INVENTION

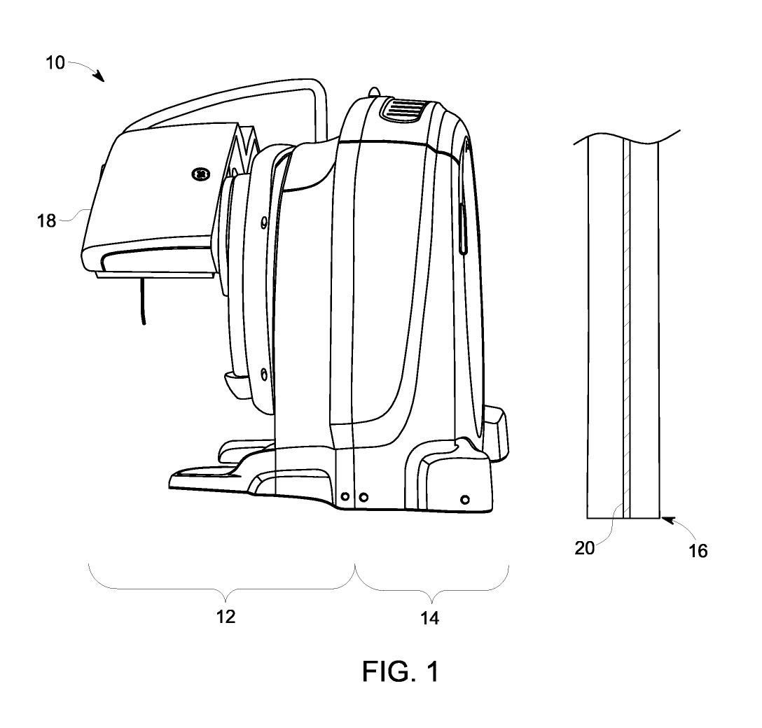

A diagrammatic representation of an exemplary SPECT/CT imaging system is shown in FIG. 1. The multi-modality system, designated generally by the reference numeral 10, is designed to acquire both structural (e.g., CT) and functional (e.g., SPECT) image data during an imaging session. In the depicted embodiment, the multi-modality imaging system 10 includes a SPECT subsystem 12 and a CT subsystem 14. As will be appreciated, though a SPECT imaging modality is primarily discussed herein, other nuclear medicine imaging modalities (such as positron emission tomography (PET)) may also be used to provide functional imaging in conjunction with the CT imaging subsystem discussed herein. It also should be noted that the rotating, dual-detector, L-mode gamma camera depicted herein is to be viewed as a non-limiting example. Other gamma camera configurations such as fixed multiple pinhole configurations or swiveling heads may be used within the scope of the invention. Additionally, the relative positioning of the two modalities may vary.

In an imaging system 10 such as the depicted SPECT/CT imaging system the subject is positioned relative to the system 10 using a patient support, e.g., a bed or table (not seen in the figure for drawing clarity). The support may be movable within the scanner to allow for imaging of different tissues or anatomies of interest within the subject. Prior to image data collection, a radioisotope, such as a radiopharmaceutical substance (sometimes referred to as a radiotracer), is administered to the patient, and may be bound or taken up by particular tissues or organs. Typical radioisotopes include various radioactive forms of elements that emit gamma radiation during decay. Various additional substances may be selectively combined with such radioisotopes to target specific areas or tissues of the body.

Gamma radiation emitted by the radioisotope is detected and localized using gamma detectors 18 of the SPECT subsystem 12. The gamma ray detectors 18 may be configured to rotate about the patient to acquire gamma ray emission data from a variety of radial views. The gamma ray emission data may then be read out by suitable data acquisition circuitry in communication with the gamma ray detectors 18. The gamma detectors 18 may be coupled to system control and processing circuitry. This circuitry may include a number of physical and functional components that cooperate to allow the collection and processing of image data to create the desired SPECT images.

Proximate to the SPECT subsystem 12, the CT subsystem 14 may be deployed to allow acquisition of structural (e.g., anatomic) image data of the region of interest near in time or concurrently with acquisition of the functional image data. The CT subsystem 14 may include a source of X-ray radiation (e.g., an X-ray tube or solid state X-ray emission component) as well as a detector component for measuring the attenuation of the emitted X-ray radiation by the patient. As discussed herein, both the source and detector of X-ray radiation may be mounted on a gantry to facilitate moving the source and detector about the patient. The detector component may communicate with detection and acquisition circuitry and downstream processing circuitry to allow the collection and processing of image data and to create the desired CT images.

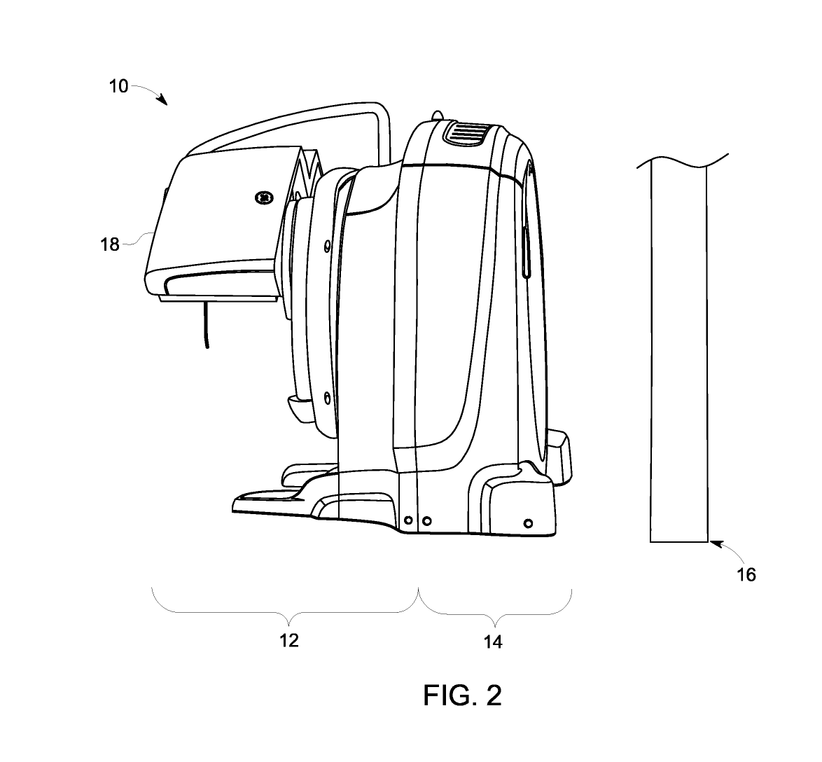

In FIG. 1, a wall 16 is also depicted representing the wall of a room in which the system 10 is deployed. In FIG. 1, the system 10 is depicted as being deployed in an existing facility, where the wall 16 may be sized to limit radiation exposure outside the room and/or may include radiation shielding 20, such lead plating having a thickness of 2 mm or more. However, while the system 10 may be used in an existing room with shielded or reinforced walls, as depicted in FIG. 1, the present system 10 may also be used in a room or facility with little or no shielding in the walls. For example, FIG. 2 depicts the system 10 in the context of a room in which the walls 16 have little or no shielding compared to facilities constructed for existing systems. As such the wall 16 of FIG. 2 may be thinner compared to previous walls in which CT systems were housed and/or may have little or no radiation shielding compared to such previous walls.

The various circuitry associated with both the SPECT subsystem 12 and the CT subsystem 14 may interact with control/interface circuitry that allows for control of the multi-modality imaging system 10 and its components. Moreover, the processing circuitry of one or both subsystems may be supported by various circuits, such as memory circuitry that may be used to store image data, calibration or correction values, routines performed by the processing circuitry, parameters for standard or routine scan protocols, and so forth. Finally, the interface circuitry may interact with or support an operator interface. The operator interface allows for imaging sequences to be commanded, scanner and system settings to be viewed and adjusted, images to be viewed, and so forth. The operator interface may include a monitor on which reconstructed images may be viewed.

With the foregoing in mind, in operation the SPECT/CT imaging system 10 may be employed to perform sequential image acquisitions which may be subsequently registered for viewing and/or analysis. For example, in one implementation a set of SPECT image data may be initially acquired for a region of interest of a patient using the SPECT subsystem 12. The patient may then be automatically translated a fixed amount so that the region of interest is properly positioned within the CT subsystem 14 and a set of CT image data may be acquired. The respective SPECT and CT images generated based on the acquired data may then be automatically registered based on the fixed and known translation of the patient. Alternatively, the order may be reversed such as the CT images are acquired first. In this case, CT images may be used for locating the organ of interest and position the patient for the SPECT imaging.

In an institutional setting, the multi-modality imaging system 10 may be coupled to one or more networks to allow for the transfer of system data to and from the imaging system 10, as well as to permit transmission and storage of image data and processed images. For example, local area networks, wide area networks, wireless networks, and so forth may allow for storage of image data on radiology department information systems or on hospital information systems. Such network connections further allow for transmission of image data to remote post-processing systems, physician offices, and so forth.

While the preceding provides general context for the use and construction of a SPECT/CT system in accordance with the present disclosure, aspects of the CT subsystem 14 will now be described in greater detail. To appreciate the manner in which the present CT subsystem may operate, certain examples of existing systems are initially discussed.

For example, certain types of existing CT subsystems used in SPECT/CT systems may employ relatively slow rotation of the CT gantry, such as due to the CT detector and the SPECT detector being mechanically coupled so as to rotate together, that is the CT and SPECT detectors rotate at the same speed. Rotation speed of such systems may be limited by the weight and fragility of the SPECT detectors. Such systems may generate images that exhibit motion artifacts due to patient motion (e.g., due to patient breathing or other motion) during the relatively slow CT data acquisition process. Such systems, however, may employ relatively low X-ray doses as compared to faster rotating, diagnostic CT systems.

Other types of existing CT subsystems used in SPECT/CT systems may employ what is essentially a standalone, diagnostic CT system as the CT subsystem. Such diagnostic CT systems may provide fast rotation of the CT gantry but also utilize relatively high X-ray doses. As a result, the CT images acquired using such stand-alone systems may themselves be suitable for diagnostic purposes, as opposed to just localization of the large organs and internal structures. That is, such high rotation speed, high dose systems may operate at diagnostic image quality (i.e., resolutions in the mm or sub-mm range, good contrast in Hounsfield numbers and high signal to noise ration) that are beyond what is needed for typical SPECT/CT operation. Instead, such SPECT/CT operations may work satisfactorily with just localization (i.e., position) information derived from the CT image data since such localization information may be sufficient for registration and/or attenuation correction of the SPECT image data, which provides the diagnostic information.

Therefore, in certain implementations of the present approach, a fast rotation, low dose CT subsystem is employed as part of a SPECT/CT imaging system. For example, one embodiment of such a system rotates the CT detector and X-ray source at about 60 RPM (i.e., faster than the gamma ray detecting components of the SPECT subsystem 12) while achieving a dose associated with low dose slow rotating CT (about 10-20 mAs). In such an embodiment, a conventional X-ray detector may be employed, though with dynamic range calibration suitable for the low dose implementation. Further, due to the relatively low dose usage, the CT subsystem and/or the room housing the CT subsystem may employ little or no shielding, especially in comparison to diagnostic level CT systems. For example, in one such embodiment, the CT subsystem 14 may be employed in a room in which the walls do not include lead or other shielding materials.

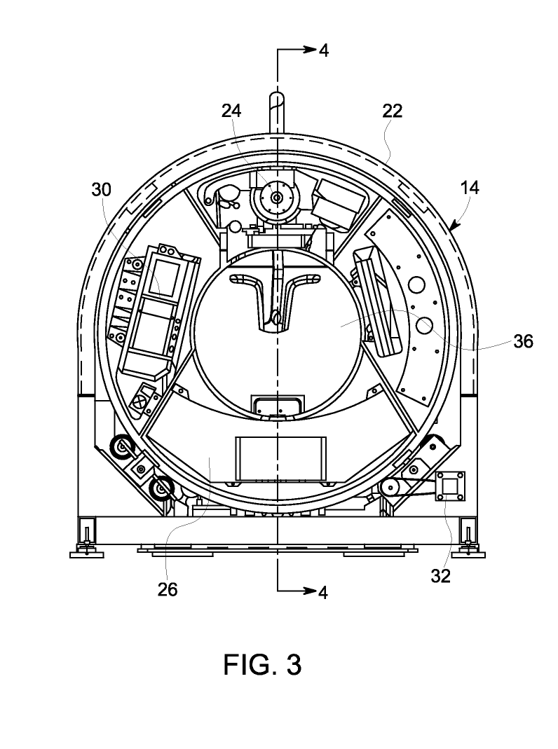

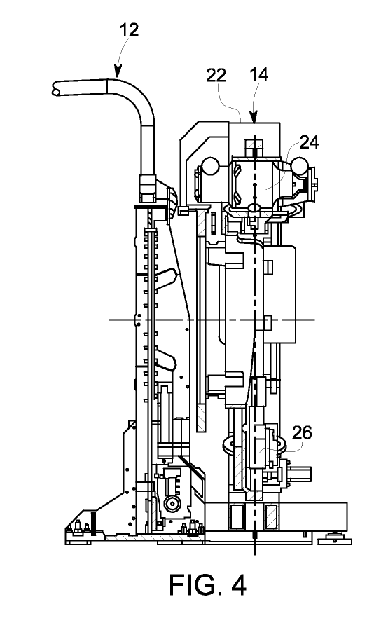

Turning to FIGS. 3 and 4, an example of a fast rotation (e.g., at or above 30 RPM, such as about 60 RPM), low dose (e.g., about 10-30 mAs) CT subsystem 14 is depicted. In the depicted example, the SPECT subsystem 12 and CT subsystem 14 are mechanically and/or operationally coupled and are not simply standalone systems brought into proximity with one another.

In the depicted implementation of FIGS. 3 and 4, the CT subsystem 14 includes a gantry 22 that provides the rotational framework for those components of the CT subsystem 14 that rotate with respect to the patient. These rotating components may include, but are not limited to an X-ray source or tube 24 and a data measurement system, (e.g., detector 26). A high-voltage generator 30 may provide power to one or more components of the CT subsystem 14, such as the X-ray source 24. In the depicted embodiment, little or no additional shielding is provided on the CT subsystem 14 (or in the surrounding environment or room, as depicted in FIG. 2) due to the relatively low dose of X-rays that the CT subsystem 14 is configured to employ. The optional reduced shielding may reduce the weight of the CT rotor, saving space, cost and complexity. Additionally, high-voltage generator 30 and X-ray source 24 may be adapted to operate at reduced power, and can thereby operate with less heat removal, further reducing weight of the CT rotor, saving space, cost and complexity.

Further, the depicted implementation of CT subsystem 14 also has a slim profile compared to stand-alone or diagnostic type CT systems. For example, in one embodiment, the CT subsystem 14 may be approximately 70 inches wide (for example, 69.5 inches or approximately 176.53 cm), approximately 75 inches high (for example, 73.73 inches or approximately 187.27 cm), and approximately 20 inches (for example, 18.38 inches or approximately 46.69 cm) from the scan plane of the CT subsystem 14 to the bearing mating with the SPECT subsystem 12. Such an example of a CT subsystem may have a bore size of 700 mm (e.g., diameter of bore 36) and provide a field of view of approximately 500 mm. In one embodiment a 4-slice detector 26 is employed where each slice has a slice thickness of 2.5 mm, providing 10 mm of axial coverage at isocenter. In such an embodiment, each detector slice may have upwards of 500 physical detectors per slice (e.g., 544 physical detectors per slice or higher).

The X-ray source 24 employed in such an implementation may be configured to operate between at a maximum 30 mA and a minimum 10 mA. Further, such an embodiment may operate at a maximum 140 kV with respect to the X-ray source 24. Where the CT subsystem achieves a rotation speed of 1 second (i.e., 60 RPM), a 40 cm scan time may be achieved in 26 seconds for a helical scan (assuming 2.5 mm per detector slice and 1.5 pitch) or 40 seconds for an axial scan.

In operation, one implementation of the CT subsystem 12 as discussed herein may be employed to obtain fast rotation, low dose CT images generally suitable for localization of internal images or structures, but not for diagnostic image review (i.e., the images do not have mm or sub-mm resolution, high SNR and high contrast). Due to the low dose associated with the CT subsystem 14, additional shielding may not be employed in the CT subsystem 14 or surrounding environment, while, due to the fast rotation speed (i.e., approximately 60 RPM) motion artifacts may be reduced or eliminated in the CT images compared to CT systems rotating at slower speeds. In this manner, CT images may be acquired using the CT subsystem 14 that have reduced or no motion artifacts but which also provide sufficient image quality for localization or attenuation correction of the internal organs or structures, and for attenuation correction of the SPECT image, without providing diagnostic level image quality or detail. Further, the known and fixed translation of the patient in the combined SPECT/CT imaging system 10 may allow the images acquired using the CT subsystem 14 to be readily registered to the images acquired by the SPECT subsystem 12.

Further, though the context of a SPECT/CT imaging system 10 is discussed, it should be appreciated that a CT subsystem 14 having characteristics as discussed herein may be used in a variety of other contexts. For example, a CT system 14 having fast rotation, low dose, and generating images of less than diagnostic image quality may be used in a emergency room or triage context, where a rapid, high level view of the internal structures of a patient may be useful in quickly determining a course of action, but is not used primarily in a diagnostic sense. Likewise, such a CT system 14 may be useful in a surgical navigation context or a minimally invasive surgery context, such as for providing preliminary organ positions and/or for tracking an interventional instrument (e.g., a stent or catheter) in a patient. Similarly, such a CT system 14 may be useful in radiation therapy planning and patient positioning. Optionally, such a CT system 14 may be used, for example in emergency, surgery or intensive care setting while medical personnel remains in the vicinity of the patient and need not move to a shielded location for the duration of the CT exposure. Additionally, such a CT system 14 may be made mobile and moved to the patient's location due to its reduced power requirement, reduced weight and the absence of shielding.

Technical effects of the invention include the use of a low dose, fast rotation CT system in the acquisition of non-diagnostic CT images with no or few patient motion related image artifacts. Examples of such systems may rotate at about 60 RPM and/or may generate a dose consistent with X-ray generation at 20 mA. Non-diagnostic images may be generated in this manner that are suitable for registration and/or attenuation correction, but which do not have the image quality generally associated with diagnostic reviews and/or analysis.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A dual-modality imaging system, comprising:
a nuclear medicine imaging subsystem comprising a gamma ray detection component suitable for acquiring functional image data; and
a computed tomography (CT) subsystem suitable for acquiring structural image data, wherein the CT subsystem comprises a gantry housing an X-ray source and an X-ray detector that are configured to rotate with respect to the gantry, wherein the X-ray source and the X-ray detector rotate above 30 revolution per minute (RPM) during operation, and wherein the X-ray source operates at a current level below 30 mA during operation.

2. The dual-modality imaging system of claim 1, wherein the nuclear medicine imaging modality comprises one of a single photon emission computed tomography (SPECT) system or a positron emission tomography (PET) system.

3. The dual-modality imaging system of claim 1 wherein the nuclear medicine imaging subsystem and the CT subsystem are one or both of mechanically or operationally coupled to form the dual-modality imaging system.

4. The dual-modality imaging system of claim 1, wherein the CT subsystem has an associated footprint of about 70 inches by 20 inches.

5. The dual-modality imaging system of claim 1, wherein a room in which the CT subsystem is housed does not include radiation shielding.

6. The dual-modality imaging system of claim 1, wherein the X-ray source operates at about 20 mA.

7. The dual-modality imaging system of claim 1, wherein the X-ray detector of the CT subsystem rotates faster than the gamma ray detection component of the nuclear medicine imaging subsystem when in operation.

8. The dual-modality imaging system of claim 1, wherein the CT subsystem has a thickness of 20 inches or less.

9. The dual-modality imaging system of claim 1, wherein the CT subsystem generates images that do not have diagnostic image quality.

10. A dual-modality imaging method, comprising:
acquiring a set of functional image data using a nuclear medicine imaging subsystem of a dual-modality imaging system;
acquiring a set of computed tomography (CT) imaging data using a CT imaging subsystem, wherein a detector of the CT subsystem rotates at least above 30 revolutions per minute (RPM) and an X-ray source of the CT subsystem operates at a current between about 10 mA and about 30 mA during acquisition of the set of CT imaging data; and
generating a localization image or attenuation map using the set of CT imaging data.

11. The dual-modality imaging method of claim 10, comprising registering the localization image with a function image generated from the set of functional image data.

12. The dual-modality imaging method of claim 10, wherein acquiring the set of functional image data comprises acquiring a set of single photon emission computed tomography (SPECT) data or a set of positron emission tomography (PET) data.

13. The dual-modality imaging method of claim 10, wherein the set of functional image data and the set of CT imaging data are acquired sequentially.

14. The dual-modality imaging method of claim 10, comprising translating a patient a fixed distance such that a specified region of interest is imaged during both the acquisition of the set of functional image data and the acquisition of the set of CT imaging data.

15. The dual-modality imaging method of claim 10, wherein the localization image does not have mm or sub-mm resolution.

16. A CT imaging system, comprising:
a gantry;
an X-ray detector configured to rotate about the gantry; and
an X-ray source configured to rotate about the gantry, wherein the X-ray source operates at a current level of between about 10 mA and about 30 mA during operation;
wherein the X-ray source and the X-ray detector during operation rotate about the gantry at above 30 revolutions per minute (RPM).

17. The CT imaging system of claim 16, comprising detector acquisition circuitry configured to generate one or more images from signals generated by the X-ray detector, wherein the one or more images are at a non-diagnostic image quality.

18. The CT imaging system of claim 16, wherein the CT system has an associated footprint of about 70 inches by 20 inches.

19. The CT imaging system of claim 16, wherein a dynamic range of the X-ray detector is calibrated for use at low dose levels.

20. The CT imaging system of claim 16, wherein the CT imaging system is used in one or more of a dual-modality imaging context, a surgical navigation context, or an emergency room context.
